(19) United States
(12) Patent Application Publication
(10) Pub. No.: US 2022/0183880 A1
(43) Pub. Date: Jun. 16, 2022

(54) METHOD AND APPARATUS TO MODIFY THE CORNEA USING ELECTROCHEMISTRY

(71) Applicants: THE REGENTS OF THE UNIVERSITY OF CALIFORNIA, OAKLAND, CA (US); OCCIDENTAL COLLEGE, LOS ANGELES, CA (US)

(72) Inventors: BRIAN JET-FEI WONG, IRVINE, CA (US); MICHAEL G. HILL, PASADENA, CA (US)

(73) Assignees: THE REGENTS OF THE UNIVERSITY OF CALIFORNIA, OAKLAND, CA (US); OCCIDENTAL COLLEGE, LOS ANGELES, CA (US)

(21) Appl. No.: 17/310,831

(22) PCT Filed: Apr. 2, 2020
(86) PCT No.: PCT/US20/26414

§ 371 Date: Aug. 25, 2021

Related U.S. Application Data
(60) Provisional application No. 62/828,276, filed on Apr. 2, 2019.
(60) Provisional application No. 62/901,487, filed on Sep. 17, 2019.

(51) Int. Cl. A61F 9/00; A61F 9/013; A61N 1/30; A61N 1/04

(52) U.S. Cl. CPC A61F 9/0008; A61F 9/013; A61N 1/0432; A61F 9/0017; A61N 1/303

(57) ABSTRACT

A method of altering corneal tissue includes creating an electrochemical reaction in the tissue, wherein the electrochemical reaction occurs in the presence of an electrolytic solution in or on the tissue.

### CROSS REFERENCE TO RELATED APPLICATIONS

This application claims the benefit of and priority to PCT application no. PCT/US2020/026414, filed Apr. 2, 2020; US application nos. 62/828,276 filed Apr. 2, 2019; and 62/901,487 filed Sep. 17, 2019, all of which are incorporated herein in their entirety.

### BACKGROUND OF THE INVENTION

The present invention generally relates to the treatment and/or alteration of corneal tissue and, more particularly, to apparatus and methods of treating and/or altering corneal tissue using electrochemistry.

The cornea is a clear and highly organized anatomical structure of the anterior eye that both protects the integrity of intraocular structures and provides the majority of the refractive power of the eye.⁵ The corneal stroma consists of orthogonally stacked collagen-fibril lamellae whose molecular composition and precise macromolecular geometry eliminate backscattered light and maintain the shape of the cornea. Anatomical variation, birth defects, trauma, and various corneal dystrophies can alter the shape and transparency of the cornea, thus affecting vision.

For example, in the United States, myopia and high myopia affect over 40% of the population.^{1,2} Significantly, high myopia is associated with several retinal degenerative conditions (e.g., myopic traction maculopathy), as well as retinal detachment and associated complications.⁶ Myopia also correlates with an elevated risk of keratoconus, a thinning/bulging of the cornea.⁷ Keratoconus is the most common corneal dystrophy, affecting ˜1 in 2000 people, mostly teenagers and young adults, resulting in roughly 7,000 corneal transplants annually.

Current interventions for these conditions pose serious challenges in accessibility, effectiveness, and/or long-term safety. For example, the standard of care for surgical correction of myopia, hyperopia and astigmatism is corneal photoablation, i.e., LASIK (laser-assisted in situ keratomileusis) or PRK (photorefractive keratotomy). Significantly, both of these procedures permanently reduce the biomechanical stability of the cornea, putting the patient at risk of developing post-treatment ectasias resulting from a weakened corneal structure.³ Other side effects include excessive glare and “halos” associated with diminished night-vision acuity.⁸ Less aggressive ablation procedures such as laser-assisted subepithelial keratectomy (LASEK) that target superficial corneal stroma may be suitable for a larger patient base, but still remain prohibitively expensive for many potential candidates (the average cost of laser-based intervention ranges between $1,500-$3,000 per eye).

Non-surgical therapies also have significant downsides. For example, orthokeratology temporarily changes the refractive power of the eye by bending the corneal surface via hard contact lenses worn at night. Drawbacks of this strategy include: a long treatment period (typically weeks) to achieve near-emmetropic acuity; the requirement for nightly installation of “retainer” lenses to prevent shape recidivism; and elevated risks of bacterial, protozoan, and herpetic keratitis.^{9,10,11} Indeed, a recent trial involving 122 subjects over a 9-month period resulted in 83 subjects dropping out of the study, highlighting the compliance challenges posed by this technology.¹²

Newer technologies based on photochemically crosslinking the cornea to affect shape change have been recently proposed. These create altered tissue that poses problems for proper healing following any subsequent trauma, e.g., corneal abrasions. However, they do represent a class of non-surgical corneal shape change, albeit often with the application of a drug.

Even in light of these considerations, vision-refraction therapy remains extremely popular, with more than 700,000 LASIK procedures carried out in the United States in 2019. Clearly there is a public health need for a less-expensive, less-invasive, and safer modality for vision refraction.

As can be seen, there is a need for improved apparatus and methods of treating corneal disease.

### SUMMARY OF THE INVENTION

In one aspect of the invention, a method of altering corneal tissue comprises creating an electrochemical reaction in the tissue; wherein the electrochemical reaction occurs in the presence of an electrolytic solution or gel in or on the tissue.

In another aspect of the invention, a method of altering corneal tissue comprises using at an anodic electrode and/or a cathodic electrode in contact with the tissue to initiate an electrochemical reaction in the tissue; and/or adding an electrolytic solution or gel to the tissue by application, irrigation, or injection.

In a further aspect of the present invention, a method of altering corneal tissue comprises mechanically disrupting the tissue; and electrochemically modifying the tissue.

In another further aspect of the present invention, a method of altering corneal tissue comprises electrochemically generating hydrochloric acid, sodium hydroxide, hydrogen gas, and either chlorine gas or oxygen gas in the tissue; and electrochemically forming acid/base species in the tissue. Other species can be generated as well, depending upon the nature of the applied solute or gel.

In another aspect of the invention, apparatus for altering corneal tissue comprises an electronic system configured to cause an electrochemical reaction in the tissue; wherein the electrochemical reaction occurs in the presence of native tissue water or an electrolytic solution or gel, which may or may not be water based.

In another aspect of the present invention, an apparatus to reshape the corneal tissue comprises means for creating stress in the tissue to temporarily define and maintain a predetermined shape of the tissue; means for monitoring the internal stresses, geometry, pH, optical clarity, acoustic properties, mechanical properties, and temperature of the tissue; means for causing a direct current of a predetermined polarity to flow in the tissue to mediate the tissue while the created stress is present to permanently change shape of the tissue or material parameters of the tissue without ablation or carbonization; and means for controlling the direct current flowing in the tissue according to the stresses therein.

In another aspect of the present invention, an apparatus of reshaping tissue to reshape the tissue comprises means for creating stress in the tissue to temporarily define and maintain a predetermined shape of the tissue; means for causing a direct current of a predetermined polarity to flow in the tissue; means for applying voltage pulses of the same or opposite polarity to form a DC pulse train to mediate the tissue while the created stress is present to permanently change shape of the tissue or material parameters of the tissue without carbonization or ablation; means for applying a voltage of predetermined polarity to obtain a predetermined bioeffect; means for applying a first sequence of voltage pulses of the same polarity and means for applying a second sequence of voltage pulses of the opposite polarity or same polarity with different magnitude to form a complex DC pulse train.

In yet another aspect of the present invention, apparatus for altering corneal tissue comprises at least two electrodes configured to cause an electrochemical reaction in the tissue; wherein the electrochemical reaction occurs in the presence of an electrolytic solution or gel; and a controller in communication with the electrodes and configured to: create an electrical potential across the electrodes; and cause an oxidation reaction spatially distinct in the tissue from a reduction reaction in the tissue.

In a further aspect of the present invention, a computer-implemented method for altering corneal tissue comprises initiating, by a processor, an electrochemical reaction in the tissue; wherein the electrochemical reaction occurs in the presence of an electrolytic solution or gel in the tissue.

In a still further aspect of the present invention, a non-transitory computer readable medium with computer executable instructions stored thereon, executed by a processor, to perform a method for altering corneal tissue, the method comprising inducing an electrochemical reaction in the tissue; wherein the electrochemical reaction occurs in the presence of an electrolytic solution or gel in the tissue.

These and other features, aspects and advantages of the present invention will become better understood with reference to the following drawings, description and claims.

### BRIEF DESCRIPTION OF THE DRAWINGS

FIG. 1a-1c are photographs of in vivo cartilage of rabbit ear according to the present invention.

FIG. 2 is blank.

FIG. 3 is a depiction of a cornea.

FIG. 4 is a depiction of an eye mount and lens support according to the present invention.

FIG. 5 is a depiction of components for lens fabrication according to the present invention.

FIG. 6 are photographs of an eye undergoing treatment according to the present invention.

FIGS. 7A-7B are SD-OCT images of a cornea according to the present invention.

FIGS. 8A-8B are photographs of a cornea and moulage according to the present invention.

FIG. 9 is a depiction of apparatus for lens fabrication according to the present invention.

FIGS. 10A-10C are depictions of a lens and cornea according to the present invention.

FIG. 11 is blank.

FIGS. 12A-12B are graphs of pH versus distance according to the present invention.

FIG. 13 is a graph of pH landscape according to the present invention.

FIG. 14 is another graph of pH landscape according to the present invention.

FIG. 15 is blank.

FIGS. 16A-16K are depictions of alternate embodiments of electrodes of the present invention.

FIG. 17 is a block diagram of a system in accordance with an embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features. However, any single inventive feature may not address any of the problems discussed above or may only address one of the problems discussed above. Further, one or more of the problems discussed above may not be fully addressed by any of the features described below.

As used herein, the abbreviation “PDEMT” refers to potential driven electrochemical modification of tissue.

As used herein, the abbreviation of “ELF” means electrochemical reshaping of tissue which can incorporate one or more aspects of electrochemical reshaping and/or electromechanical reshaping (EMR).

Broadly, the present invention provides electro-mechanical reshaping (EMR). Because EMR does not permanently alter the underlying structure of the stroma, it poses a limited risk profile for ectasia and scaring. In addition to non-surgical refraction, EMR is a potential adjunct therapy for keratoconus.

Specifically, photochemical collagen crosslinking (CXL) is FDA-approved as a method to increase corneal stiffness to prevent keratoconus progression.¹³ In a combination therapy, patients could undergo EMR to “dial in” the corrective corneal curvature followed by CXL to “lock in” the shape. PRK and CXL are currently used in conjunction to treat keratoconus in Europe. EMR could offer a similar benefit to PRK without many of the risks associated with PRK, including delayed epithelial wound healing, infectious keratitis, corneal haze formation, and further weakening the stromal biomechanics.

Similarly, penetrating keratoplasty (PK) could be optimized by EMR: following corneal transplant and suture removal. EMR could provide non-invasive post grafting refractive correction to match the shape of the donor cornea to the needs of the receiver. The successful development of corneal EMR would enable highly controlled and permanent reshaping of the cornea while conserving the integrity of its complex underlying molecular structure.

Broadly, the present invention provides EMR that relies on short electrochemical pulses to electrolyze water, with subsequent diffusion of protons into the extracellular matrix of collagenous tissues; protonation of immobilized anions within this matrix disrupts the ionic-bonding network that provides structural integrity. This leaves the tissue transiently responsive to mechanical remodeling; subsequent re-equilibration to physiological pH restores the ionic matrix, resulting in persistent shape change of the tissue. Pairing EMR with a customizable corneal reshaping contact lens thus offers the possibility of a molecular-based method to alter corneal curvature that does not require ablation of the native stromal tissue.

Broadly, the present invention provides a “molecular-surgical” modality for reshaping cornea as a safer and low-cost alternative to ablation and other laser-based refractive interventions. Localized pH gradients generated at the surface of the cornea or even in the stroma via short electrochemical pulses transiently soften the stroma, enabling remodeling of the cornea for effective vision refraction.

Broadly, the present invention provides electrochemical reshaping of corneal tissue and other related tissues with introduction of electrolytes onto or into tissues for the purpose of cosmetic or plastic surgery or for other medical treatments.

Generally, in this invention, electrolytic solution or gel is injected into and/or onto the corneal tissues that need to be treated, and needle electrodes are placed onto and/or into corneal tissues. The needles are connected to an electrical power source—as simple as a battery—which triggers chemical reactions around the needles that result in the production of hydrogen gas (at the cathode) and oxygen gas (at the anode). These reactions also raise and lower the pH in vicinity of the respective electrodes.

By employing a conventional potentiostat—an electrical circuit based on an inexpensive operational amplifier—to control the electric fields, it is possible to monitor and control precisely the quantities of acids and bases produced. By enabling control over the applied voltages, the potentiostat allows selection of specific electrochemical reactions with tight spatial resolution. Both acids and bases can hydrolyze or otherwise chemically modify corneal tissue. This invention can be useful in removal or sculpting of corneal tissue. Likewise a galvanostat can be used.

The present invention can be implemented for the treatment, shaping, and/or removal of corneal tissue. For electrochemical reactions to occur, the milieu in which the reactive species reside within tissues must conduct electrical current.

To overcome this challenge, this invention can incorporate the injection of an electrolyte solution or gel—most commonly normal saline—into and/or onto the corneal tissue. In embodiments, the electrolytic solution may contain one or more amphiphilic compounds. The injection may occur before or during the application of the electrical potential. This can be combined with a local anesthetic as well to reduce or eliminate pain associated with electric current. The injection or application of saline solution or a gel results in a change in the electrical impedance of this tissue and allows the flow of charge from anode to cathode. With the establishment of the appropriate electrical potential, water then undergoes electrolysis. Reactive species are generated, the most important being hydronium (protons) and hydroxyl ions. Non-aqueous media may be used as well as different solutes.

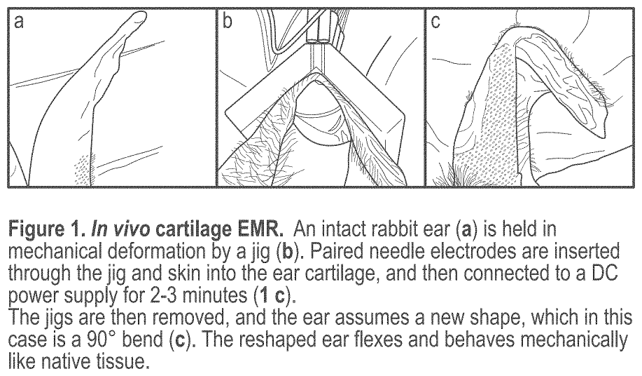

Conventional surgical intervention relies on mechanically cutting, carving, morselizing, and/or suturing collagenous tissues, typically under general anesthesia, to achieve a desired form factor. In contrast, the present invention using EMR combines mechanical deformation with the application of electric fields: in a typical embodiment, tissue is held in mechanical deformation by a mold, needle electrodes are inserted, and a constant voltage is applied across the specimen for several minutes. When the electrodes and mold are removed, the tissue assumes a new shape that approximates the geometry of the mold, FIG. 1.⁴

Within the corpus of alternative surgical techniques,^{16,17} the present invention is unique in that it employs electrochemical modalities to transiently alter the chemical properties of tissue, providing a reversible, molecular-based alternative to the scalpel and sutures (or for corneal procedures, the femtosecond laser).

Corneal EMR, according to the present invention, represents a paradigm shift from photoablation and other mechanical-based treatments, instead relying on precisely controlled acid/base chemistry to transiently alter the molecular composition of stromal tissue (deprotonated and charged vs. protonated and neutral, in addition to other mechanisms of action including but not limited to water bonds, local mineralization, denaturation etc.).

According to the present invention, apparatus and methods can alter the geometry of the cornea through a combination of physical processes. The cornea is a visco-elastic transparent tissue that refracts light. Placement of a firm contact lens over the cornea to which electrodes are attached would allow the application of direct current, either constant, or modulated to the corneal surface. Here cornea is referred to as a composite structure consisting of both epithelium and stroma. Likewise electrodes may be surface devices or may even penetrate, partially or full thickness through the entire corneal structure.

During the application of electrical energy, redox reactions occur. These can be controlled through a number of techniques, such as that disclosed in US application Ser. No. 14/280,524 filed May 16, 2014; Ser. No. 15/898,459 filed Feb. 17, 2018; and Ser. No. 15/900,985 filed Feb. 21, 2018, all of which are incorporated herein by reference. This includes the application of an electrical potential in a bipolar mode, or via the application of a potentiostat, or alternatively a galvanostat. When energy is applied, water in the cornea matrix undergoes electrolysis creating new chemical species in situ. There are several active species generated, though H+ and OH− are the species which we have demonstrated to have active function in terms of altering tissue collagen and glycosaminoglycan behavior.

Electrical energy electrolyses water and this alters corneal tissue shape. It may be necessary to wear a second contact lens after therapy, in order to stabilize shape, and/or guide the remodeling process, and these lens may be required for a variable time, and may progress through a variation of different shapes over time.

The geometry of the tissue-electrode interface can be designed in a number of different ways, incorporating the printing of electrodes on the inner surface of a rigid or soft contact lens. The lens may also have electrodes micro-machined, etched (lithography), printed, or simply bonded, and these electrodes may have complex shape in three dimensions. Electrodes may be simple with anode and cathode pairs, or complex with even multiple components.

Incorporation of a potentiostat or galvanostat may involve the use of a working, counter, and reference electrode systems. The geometry of each electrode need not be identical or symmetric, and anode/cathode may differ from one another in design.

A second direct application of the invention is that electrical potentials to the cornea results in changes in tissue composition. This can be used to correct corneal dystrophies produced by mechanical trauma, thermal injury (burn), light/laser, or chemical injury. This may be used to clear opacities for example.

Feedback

Monitoring of the shape change process or structural/compositional changes in the cornea can be achieved by:
measuring the potential as well as the charge transfer during the shape change process.
measuring the current applied during treatment.
monitoring temperature, though this is known not to rely upon thermal mechanisms.
optically, using both diffuse and coherent imaging methods including light microscopy, non-linear optical microscopy, OCT and other interferometric techniques.
monitoring localized changes in tissue internal stress using our acoustic and optoacoustic methods, including strain gauges and related devices, that provide information on stress and strain.
pH can be monitored using simple electrodes, dye systems, or other similar technology. Electrical device parameters can be adjusted dynamically to alter pH landscape—spatial distribution pattern.
monitoring or feedback can occur at any time before, during or after the reshaping process.

Electrodes
Electrode composition may include any conductive metal, rare earth elements, and graphite, or any other conductive material.
May be flat, raised, undulating, needle shaped, narrow, broad or curved.
Patterns can be complex shaped in 3 dimensions.
Electrodes may not require attachment to a contact lens (hard) or corneal mandrel/moulage.
Electrode may be coated with catalytic materials to facilitate electrolysis of specific substrates, namely water.

Electrical Dosimetry
Applied potential and current can be varied.
Modulation: can occur to ramp, step, or reverse polarity, or be of a complex pattern or waveform.

### Examples

From a molecular point of view, collagenous tissues are polymer hydrogels consisting of highly organized collagen fibrils surrounded by a proteoglycan matrix. Highly sulfated glycosaminoglycans (GAGs) that are deprotonated under physiological conditions provide a substantial fixed negative charge to the tissue, resulting in an ionic-bond network that provides structural rigidity. Cells that govern homeostasis and repair processes are sparsely populated within this extracellular matrix; maintaining their viability is particularly important, as inflammation following trauma can lead to unregulated production of fibrous tissue, with subsequent susceptibility to scarring and loss of function.¹⁸

Chemically damaged ex vivo eyes that result in corneal opacities have been successfully cleared by the present invention. Ex vivo rabbit cornea have also been successfully reshaped, resulting in flatter cornea (potential treatment for myopia), more curved cornea (potential treatment for hyperopia), and cornea with multiple planes of focus (potential treatment for presbyopia), according to the present invention.

Molecular mechanism of shape change. We considered several modes of action for electromechanical tissue reconstruction: (i) dehydration of the hydrogel matrix followed by tissue denaturation; (ii) electrophoresis; and (iii) chemical modification of the GAG matrix.

From these experiments, it is clear that acidification at the anode (a consequence of water oxidation) and subsequent diffusion of protons into the tissue is the dominant process responsible for shape change. Protonation of immobilized anions within the GAG matrix disrupts the ionic-bonding network that provides structural integrity to the tissue. This, in turn, relieves the stress imposed by mechanical deformation. Re-equilibration to physiological pH may restore the fixed-charge density (FCD) after molecules have locally “shifted” and reestablishes the ionic-bonding matrix, resulting in sustained shape change of the tissue. It is noteworthy that this mechanism explains our observation that EMR persists ex vivo only if the pH is re-equilibrated in neutral buffer for several minutes following electrolysis; if the specimen is instead removed from the jig immediately after electrolysis, the tissue remains malleable near the electrode placements.

Reshaping cornea presents a number of stringent design challenges that must be addressed: (i) to avoid inserting needles into the eye, corneal EMR should be applied as a surface technique; (ii) corneal EMR must exhibit extremely fine control over the tissue form factor, as a typical refractive procedure might involve remodeling the corneal surface by only a few microns; and (iii) it is imperative that keratocyte viability and the underlying stromal structure should be preserved to prevent corneal haziness and scaring. Illustrative examples for addressing some of these challenges are described.

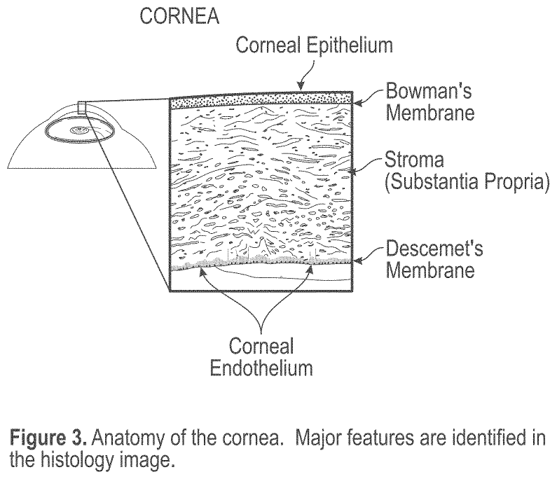

Electrode Design and Fabrication. The cornea is comprised of five layers, consisting of the epithelium, Bowman's membrane, stroma, Descemet's membrane, and endothelium (FIG. 3). The stroma is the largest of these layers and provides the tensile strength for maintaining the cornea's shape.¹⁹ Collagen molecules within the stroma are organized into uniform fibrils that span the entire plane of the structure. Transparency of corneal tissue relies on the precise lattice arrangement of these fibrils to eliminate backscattered light.²⁰ In the stroma, Type I collagen triple helices are arranged into orthogonal lamellae;²¹ the individual fibrils have a smaller diameter than in other connective tissues and the overall structure is supported by proteoglycans and Type V collagen. The central 4-mm region around the apex of the cornea is quasi-spherical while the periphery adopts a prolate ellipsoidal shape.⁵ This central region is the target of our reshaping efforts.

The reshaping process depends on the electrochemical generation of protons at the corneal surface. In principle, this could be accomplished using a simple voltage divider in series with a battery. In a DC powered circuit, there are two electrodes—the anode held at positive voltages where oxidation occurs (Eq. 2) and the cathode held at negative voltages where reduction takes place (Eq.1). When applied under conditions of high impedance however, this circuit is prone to large errors, as current passing through the tissue results in a significant potential drop across the medium. Thus, we instead carry out the reshaping process using a potentiostat/galvanostat. A potentiostat features three electrodes: the working (WE), counter (CE), and reference (RE) electrodes that are connected by a non-linear circuit (operational amplifier). The potentiostat controls and maintains a constant potential at the working electrode where redox reactions of interest occur, by applying a sufficient voltage at the counter electrode, which is in electrical contact with the WE via the surrounding electrolyte. The voltage at the CE is continually adjusted to provide a constant potential at the WE via feedback from the reference electrode. The CE may be a significant distance from the cornea surface itself, provided that electrical continuity is maintained.

Given that the working electrode needs to be in contact with the corneal surface, the question becomes how to incorporate the other two electrodes into the device. Importantly, the counter electrode effectively serves as the cathode, generating hydroxide ions during EMR treatment. In order to eliminate base-induced tissue damage, the CE can be encased within a conductive gel separated from the corneal tissue by an ion-permeable membrane. A silver-wire can serve as the reference electrode, embedded into the contact-lens mold and shaped into a ring around the WE in order to minimize potential drop across the large surface area of the WE.

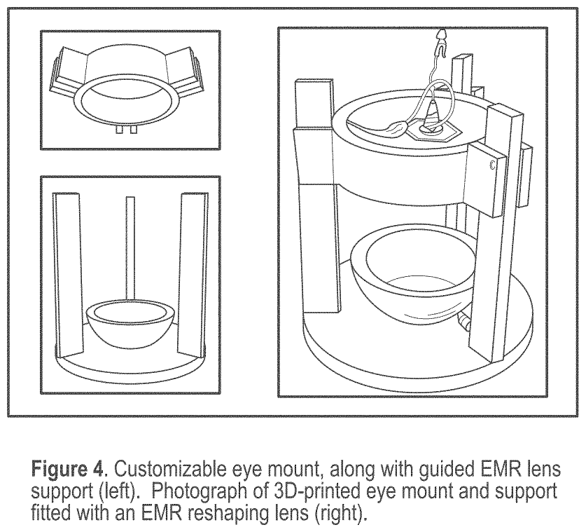

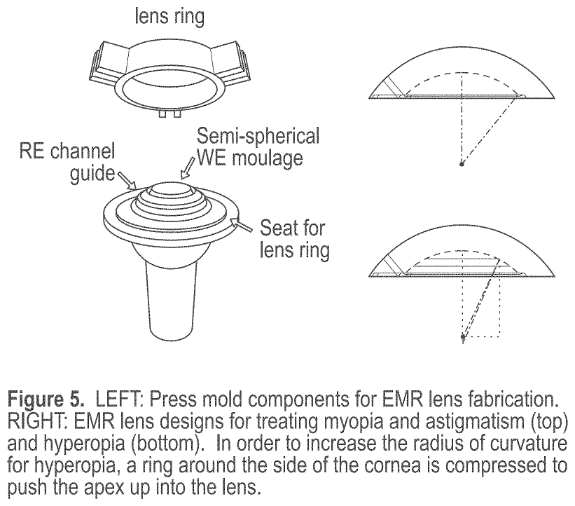

Corneal EMR jig. The illustrative work shown below was carried out on New Zealand rabbit eyes, using a custom 3D-printed eye mount and matching contact lens/electrode guide (FIG. 4). The excised eye is mounted into the cup with the cornea facing up. The contact lens is then lowered onto the cornea using guides to orient the center of the WE onto the corneal apex. Although contact-lens irrigation channels can be easily incorporated into this design for in vivo trials, for ex vivo work we simply submerge the entire apparatus into phosphate buffered saline in order to maintain tissue hydration. This configuration allows for routine testing of a series of reshaping lens designs, geometries, and dosimetry conditions, all while segregating base generation from the specimen.

Press molded lens fabrication. Press molded lenses for EMR can be manufactured by stamping a 3D printed semi-spherical “plunger” featuring a form of desired corneal curvature onto a thin platinum sheet (FIG. 5). A lens-support ring with guide rails is then placed around the plunger and filled with an epoxy resin. Upon curing, the support ring is removed from the plunger. The platinum adheres to the epoxy, yielding an EMR reshaping lens that features a concave platinum-electrode surface of specified curvature. The reference electrode is incorporated by including a raised detail that encircles the form, producing a channel in the cured lens into which a silver-wire RE can be threaded. Electrodes will be connected to the potentiostat through apertures on opposite sides of the support ring.

Using this general template, we prepared a series of lenses with variable refractive powers, capable of treating both myopia and hyperopia over a wide range (roughly ±4 diopters) of refractive error. After EMR treatment, corneal curvature and clarity is measured using spectral-domain optical coherence tomography, SD-OCT.²² Three-dimensional baseline SD-OCT volumes are acquired both pre- and post-treatment. The radius of curvature for each data set is determined using a MATLAB image processing script capable of spatial resolution.

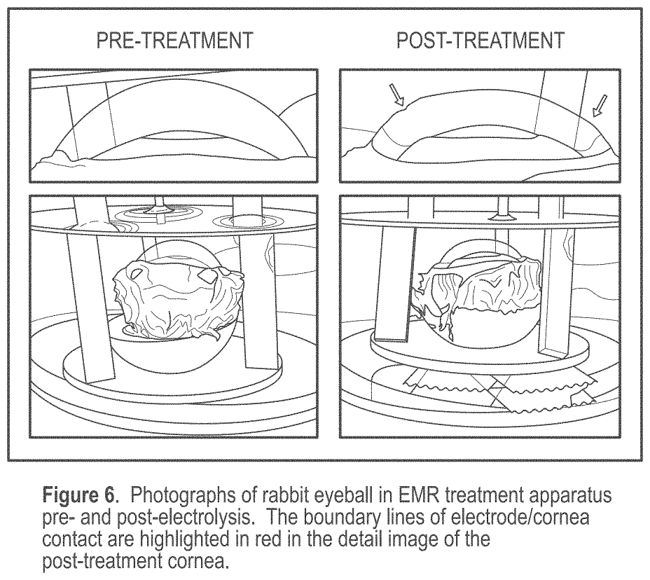

We have used this fabrication method and treatment protocol to flatten an intact rabbit cornea via EMR. FIG. 6 shows pre- and post-treatment images of an eye subjected to a 2-min EMR application (the applied potential, 1.5 V vs. AgCl/Ag, was pulsed on and off at 0.5 Hz until the total charge passed was 0.15 C). For this experiment, a reshaping lens with a 7.25 mm radius of curvature was used. The post-treatment photograph clearly shows distinct flattening of the corneal surface. (The edge effects visible in the photograph mark the boundary where the corneal surface was in direct contact with the reshaping lens.) Significantly, control experiments in which the reshaping lens is applied to the corneal surface but no electrochemical current is passed yield no changes in curvature.

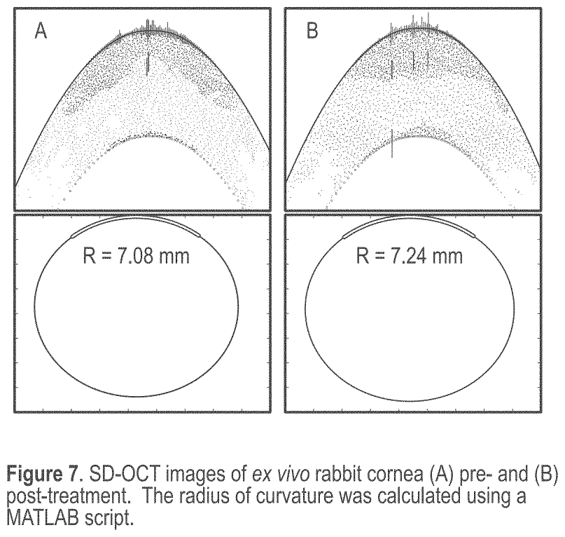

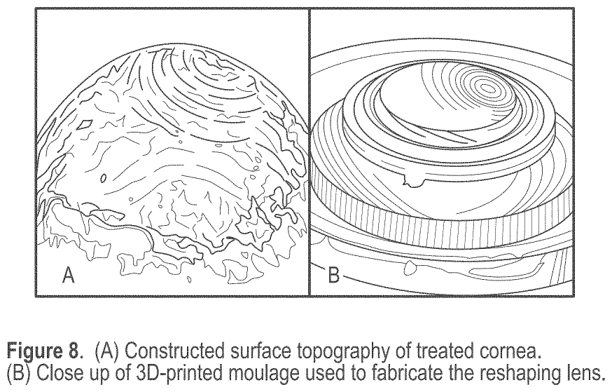

Quantitative curvature changes were measured by comparing pre- and post-treatment SD-OCT images of the cornea (FIG. 7). Notably, the curvature of the treated cornea matches nearly identically the spherical radius of the press-molded reshaping lens. Moreover, the post-treated cornea exhibits no discernable loss of transparency, and all of the anatomical features of the eye (save its curvature) appear unchanged by EMR.

To further evaluate the surface contour of the treated cornea, a series of 2D SD-OCT sections were scanned, stacked, and processed to render a 3D topographical image, illustrated in FIG. 8. The post-treatment corneal surface is uniform and qualitatively indistinguishable from an untreated specimen, with the remarkable exception of a series of shallow concentric circles inscribed onto its side. The origin of these circles can be found by examination of the 3D-printed form (FIG. 8b) used to fabricate the press-molded lens: these same features appear as vestiges of the 10-um vertical resolution of our 3D printer. Thus even subtle features of the reshaping lens appear to be readily transferred onto the corneal surface during EMR.

The ability to control so finely the corneal shape offers opportunities to explore more sophisticated lens designs to treat higher-order vision problems.

Platinum sputter-coated 3D printed lenses. Customized contact lenses of selected curvatures are produced directly using a 3D printer. Any imperfections on the concave surface will be removed by polishing with diamond paste. The polished surfaces will then be plasma treated and coated with a thin layer of polydimethylsiloxane (PDMS), which provides a robust substrate for sputter deposition of platinum.²³ This fabrication method should allow for the ready construction of more involved optical designs, including multifocal lenses. As with the press-molding design, the reshaping lens/electrode will be fitted into a guide ring that supports an embedded reference electrode. One anticipated advantage of this design is that sputter-deposited platinum, referred to as “platinum black”, is exceptionally active toward water oxidation, thus potentially shortening the time required for treatment.

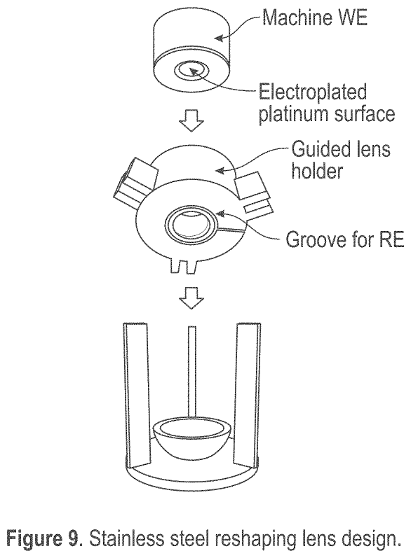

Electroplated machined stainless-steel lenses. Machined stainless steel “ingots” with specific concave curvatures also can be readily produced and used as a framework to electroplate the semi-spherical platinum WE. As platinum does not adhere well to stainless steel, electroplating a thin undercoating of nickel from a “nickel strike” solution will provide a suitable substrate for platinum electrodeposition.²⁴ Acidic solutions of chloroplatinic acid are commonly used as the deposition source.²⁵ Once formed, the electroplated ingot is inserted into a custom 3D-printed lens guide with a built-in reference electrode, as shown in FIG. 9.

Bi- and multifocal lens geometries can be prepared by stacking thin “washers” with defined spherical curvatures onto machined ingots.

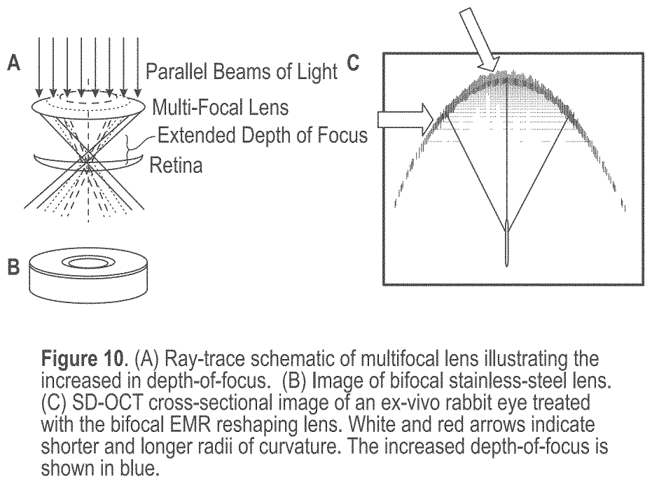

Multifocal corneal EMR. Multifocal lenses leverage concentric spatial regions of varying spherical aberration to increase the effective depth of focus of a given single lens (FIG. 10)²⁶. Each concentric area has a unique focal length that, when considered as a single optical element, extends the depth of focus. Implantable intra-ocular lenses have utilized lenses with similar multifocal properties to correct for refractive errors such as age-related presbyopia wherein light focuses behind the cornea.²⁷

Using a machined bifocal EMR reshaping lens, we have carried out a feasibility study to imprint a similar concentrically varying spherical aberration upon the corneal surface. As shown in FIG. 10c, EMR treatment induces a clear change in corneal curvature at two different radii. These results suggest that altering the corneal surface to mimic a multifocal lens is a viable application goal.

Electrochemical dosimetry optimization. One of the keys to EMR is controlled protonation of fixed negative charges within the collagen matrix, based on published work on cartilage, we estimate a pH of roughly 2 is required to decrease the stromal modulus.

Map EMR-induced pH gradients within corneal tissue. Fundamentally, EMR is a controlled acid-delivery therapy. So we must devise an EMR dosing algorithm to deliver the precise concentration of protons into the tissue necessary for shape change. A higher concentration would not improve shape change, but would increase risk of injury. The difficulty in achieving this, however, is apparent: protons are generated electrochemically at the electrode/cornea interface. As the proton flux diffuses into the tissue, [H⁺] drops off exponentially as a function of distance. As a consequence, targeting a therapeutic tissue pH at depths even just a few hundred microns from the interfacial boundary would require a far lower pH at the corneal surface.

To guide our dosimetry strategy, we have constructed a model for proton diffusion through a polyelectrolyte medium, using an analytical solution for diffusion of a protons liberated continuously from a point source into an infinite volume, Eq. 4³²:

$$C = \frac{q}{4 π D r} erfc [ \frac{r}{2 \sqrt{D t}} ] \qquad ( 4 )$$

Here C is the [H+] at a distance r from the source at time t; D is the diffusion coefficient, 7×10⁻⁵ cm²/s as estimated for Grotthuss-type diffusion through Nafion;³³ and q is the rate of proton generation at the origin.

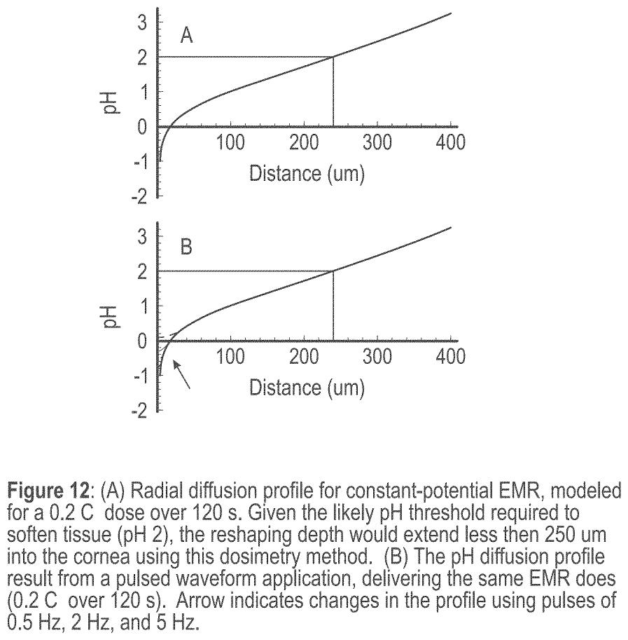

FIG. 12a presents the calculated pH gradient resulting from a constant-potential (DC) 0.2 C EMR treatment carried out over 120 seconds. Notable are two findings: (i) even using this relatively aggressive EMR dosing, the anticipated pH-threshold for tissue softening (˜2) extends only ˜250 um into the anterior face of the stroma; and (ii) electrolysis results in extremely high proton concentrations (pH<−1) near the electrode surface. We therefore considered an alternative, pulsed-potential (AC) protocol to “flatten” the pH diffusion profile. In the AC model, a defined amount of charge is deposited (a “pulse”) at the electrode for a duration that is much shorter (in our model, infinitely shorter) than the time between two adjacent pulses. This contrasts the “continuous” deposition of charge inherent to the DC approach. FIG. 12b shows a series of simulated pH diffusion profiles for different pulse rates (0.1-5 Hz), using the same 0.2 C, 120 s experimental constraints. It is immediately apparent that short dosimetry pulses have the potential to level off the pH close to the electrode surface.

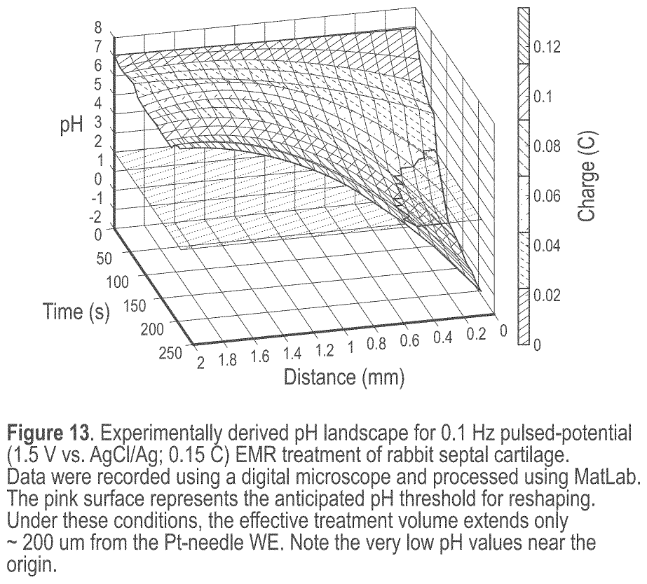

Given the importance of determining highly accurate dosimetry guidelines, we plan to develop experimental procedures to obtain empirical pH diffusion profiles as a function of EMR-dosing parameters. To test this idea, we used pH-indicating dyes to map the three-dimensional pH gradient formed during pulsed-potential EMR treatment of rabbit septal cartilage as a model. Using tissue stained with a mixture of dyes covering the 0-7 pH range, pulsed electrolysis at 0.1 Hz causes a clear color change migrating from the Pt-needle anode. This change is readily monitored with a digital camera and compared with reference color images of stained cartilage samples maintained at known acidities. As a result, the evolving pH gradient can be mapped and correlated with the charge passed. These data allow construction of an experimentally derived pH landscape as a function of distance, time, and charge, FIG. 13. Note that even with this pulsed application, the empirical pH landscape reveals a concentration of protons (low pH) near the electrode source that far exceeds the anticipated reshaping threshold. (The excellent agreement between our calculated and measured pH-diffusion maps validates our modeling approach.)

It is important to note that the depth of proton diffusion into the corneal tissue for any given EMR-treatment duration depends only on the total charge passed. Thus, while shortening the pulse time might flatten the proximal pH levels, maintaining the same EMR therapeutic tissue depth would require extending the treatment time and/or passing more charge per pulse. Extended treatment times pose obvious drawbacks from a clinical perspective, while passing more charge per pulse would require application of increasingly large electrochemical potentials. This latter approach would necessarily lead to the generation of unwanted reactive oxygen species, such as peroxide or hypochlorite—the product of chloride oxidation in aqueous media.³⁵ (ClO⁻ formation is a particularly vexing problem, as the interfacial kinetics for chloride oxidation are typically 3- to 4-orders of magnitude larger that the 4e⁻ oxidation of water at most electrode materials.) Successfully navigating these obstacles will likely require an alternative approach.

Develop pulsed chronopotentiometry with pH “leveling” for corneal EMR. Although we have successfully applied pulsed-potential dosimetry in an ad hoc way to reshape ex vivo rabbit cornea (cf. FIGS. 7-9, 11), the results shown in FIG. 13 suggest that the corneal surface pH may still drop to dangerously low levels during EMR treatment. (Such conditions apparently do not cause corneal haziness in ex vivo eyes but may affect keratocyte and/or limbal stem cell viability in vivo.). We therefore developed an alternative strategy based on pulsed chronopotentiometry. Chronopotentiometry is a standard (though less-frequently used) electrochemical technique in which the potential at the working electrode is under feedback control to produce a constant current (as opposed to a constant potential) at the interfacial boundary.³⁶ The great advantage of a constant-current application is that we can select the rate of EMR treatment, as opposed to “guessing” the appropriate potential to achieve a desired current. Moreover, we can construct a dosimetry waveform that imposes an upper limit on the applied potential at the WE in order to prevent the electrochemical generation of reactive species, e.g, HOOH or CO⁻.

Indeed, in proof of principle work, we have used a combination of pulsed chronopotentiometry and DC electrolysis to create a dosimetry algorithm that eliminates over-acidification in model systems. This strategy exploits the pH dependence of the hydrogen-evolution reaction. The thermodynamic potential for the 2e⁻ reduction of protons to H₂ is 0.00 V vs. NHE at pH 0 ([H⁺]=1M). According to the Nernst equation, the potential of this process drops by 59 mV for each unit increase in pH. Thus holding the WE potential at 0 V between pulses would limit the interfacial surface concentration to 1 M—any protons over that value would be reduced to H₂. Similarly, holding the WE at −59 mV would set the pH floor at 1; −118 mV at pH 2; −177 mV at pH 3; etc.

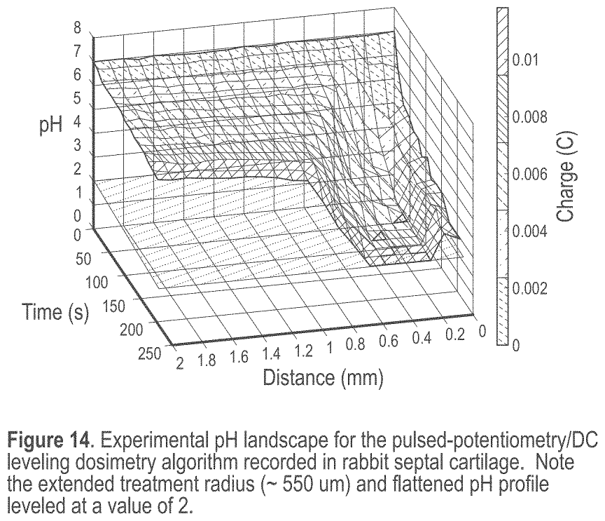

We have tested this leveling idea on articular cartilage (FIG. 14). The dosing algorithm employs short chronopotentiometric pulses at the WE. As water electrolysis proceeds, the potential at the WE increases to maintain a constant current. When the potential reaches the programed maximum (e.g., 1.5 V), the pulse is discontinued. But now, instead of allowing the WE to float at open circuit during the “off” cycle, we hold the WE at a constant potential that corresponds to the hydrogen evolution reaction at pH 2. In effect, the WE serves as a “treatment” anode during the chronopotentiometry pulses, and a “pH-leveling” cathode during the DC rest cycle. Successful, implementation of this algorithm to cornea EMR would allow us to achieve bespoke proton-diffusion landscapes, limiting corneal softening only to those stromal volumes requiring remodeling to produce a specific corneal geometry.

Post-treatment collagen structure and viability assessment. If corneal EMR is to become a viable therapy for refractive vision intervention, treatment must not compromise the cellular viability or biomechanical stability of the cornea. In Aim 3 we will establish routine assessment protocols for evaluating these parameters, The resulting data will provide crucial information to inform the iterative refinement of EMR device design and dosimetry. Following precedent from the literature, post-treatment cornea will be examined using confocal. TPM, as well as conventional histology.

Live-dead assay. In order to assess keratocyte viability following EMR, cornea will be stained with Calcein AM and ethidium homodimer-1 fluorescent dyes. Automated cell counts obtained using commercial software (e.g., MatLab, Image J, Amira, etc.) will be used to verify the number of live and dead cells. Cell viability will be collected for a range of device designs and dosing parameters, then correlated to the observed curvature-remodeling data.

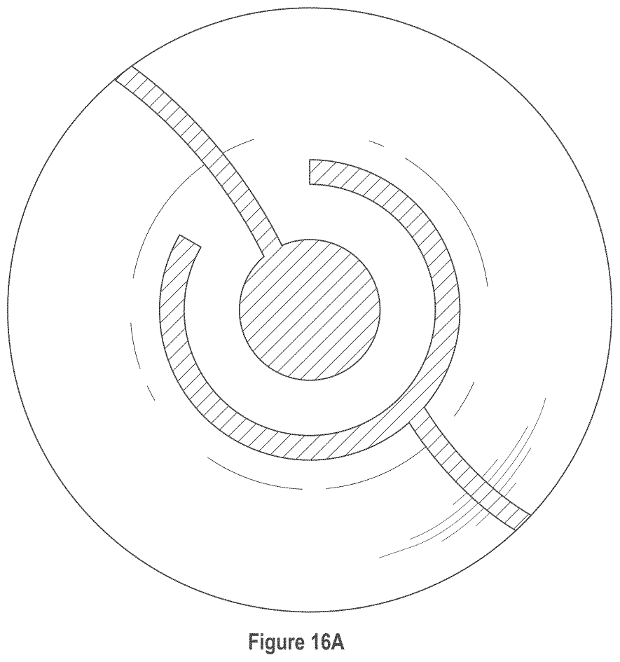

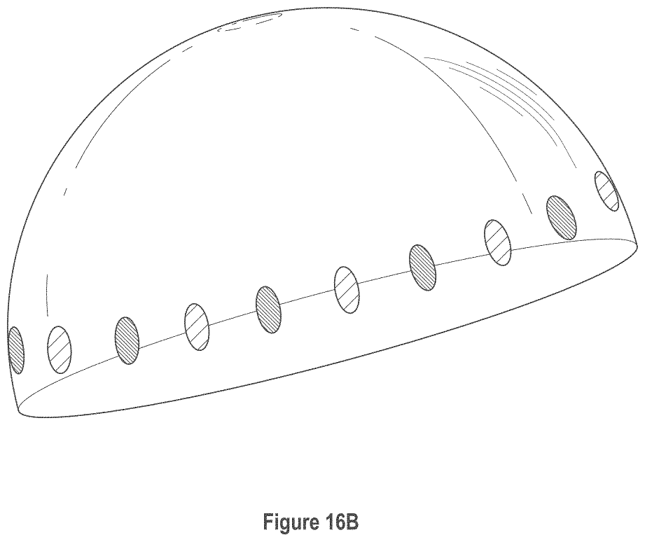

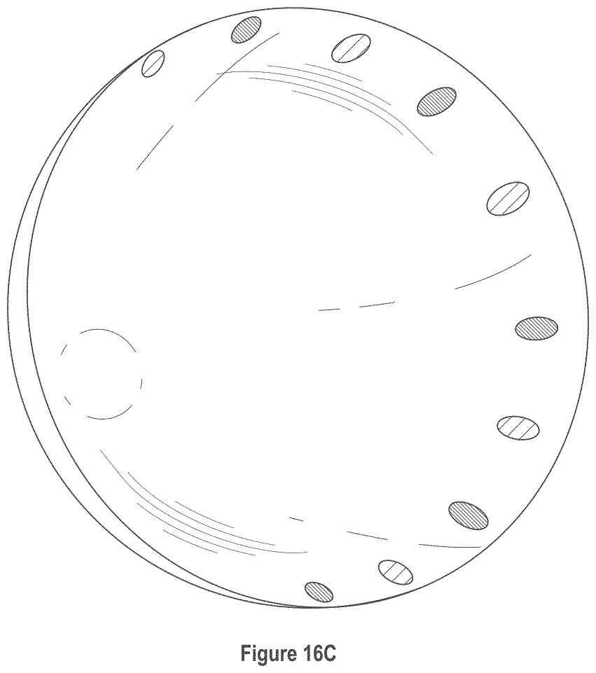

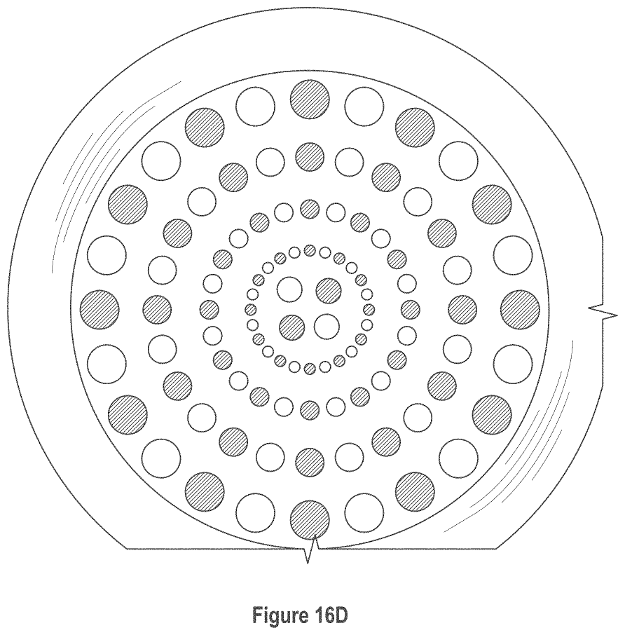

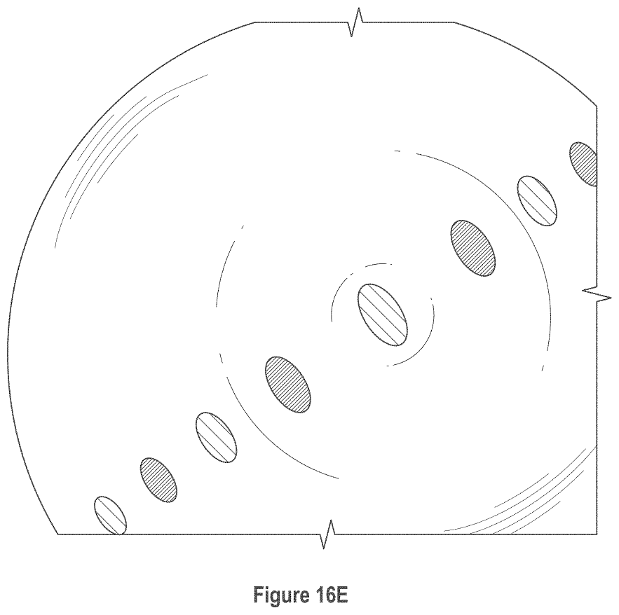

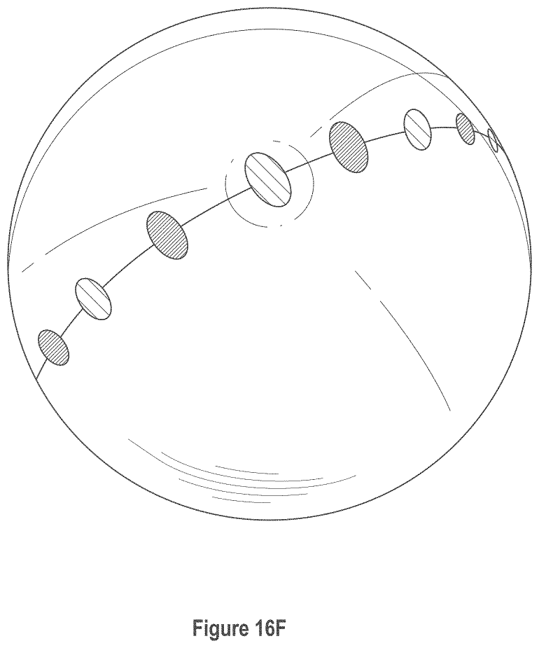

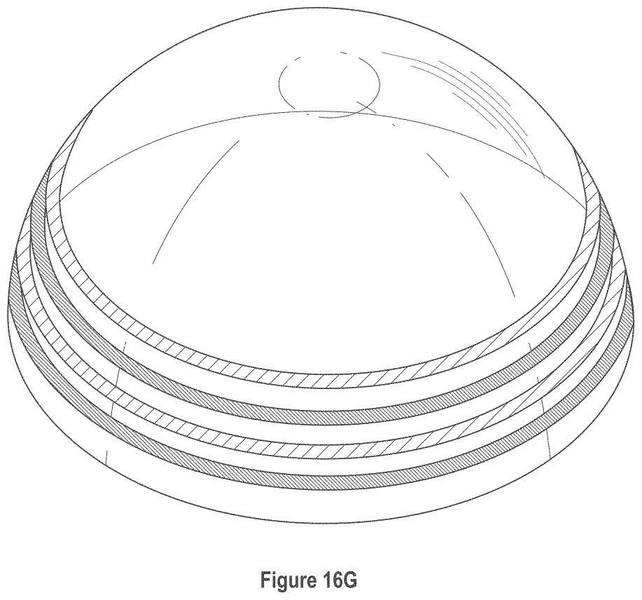

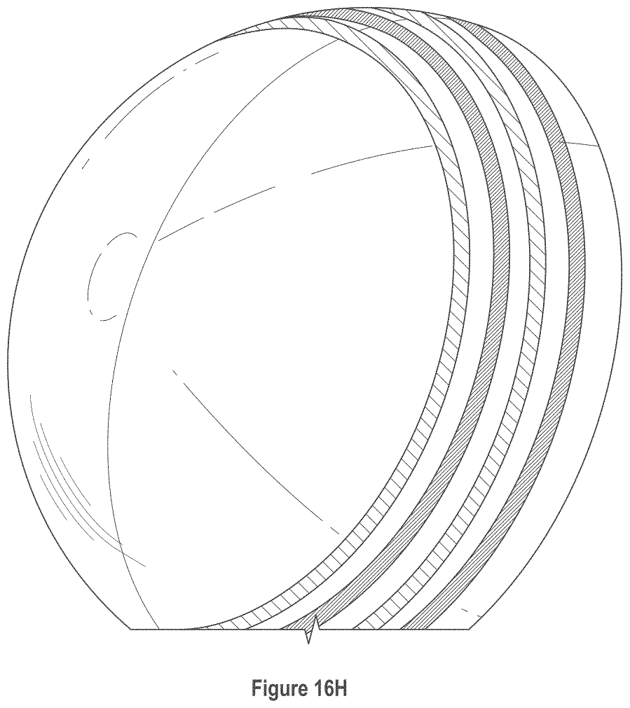

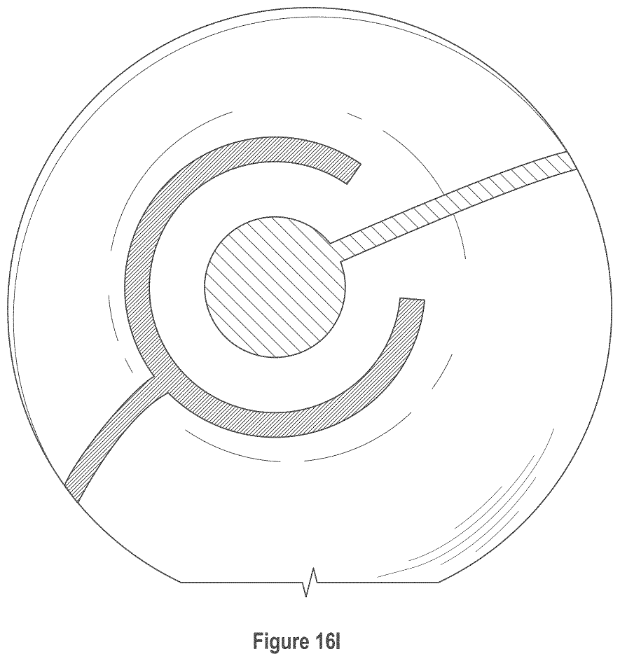

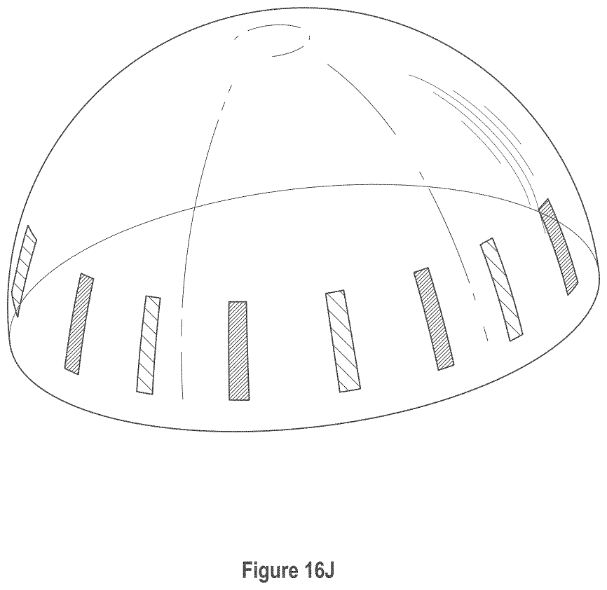

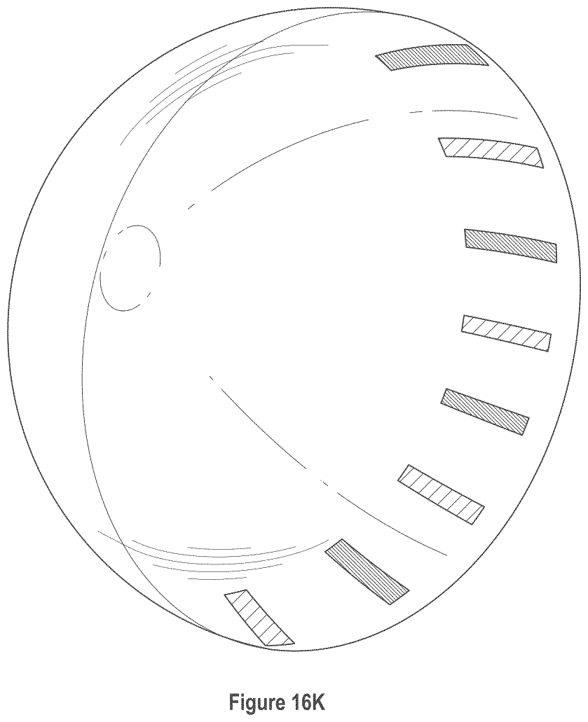

FIGS. 16A-16K are alternate embodiments of electrode arrays on and/or in a cornea in accordance with the present invention. The electrodes can be anode/cathode pairs or can be working/counter/reference electrodes. The electrodes can be of various shapes including needle, planar, and curved.

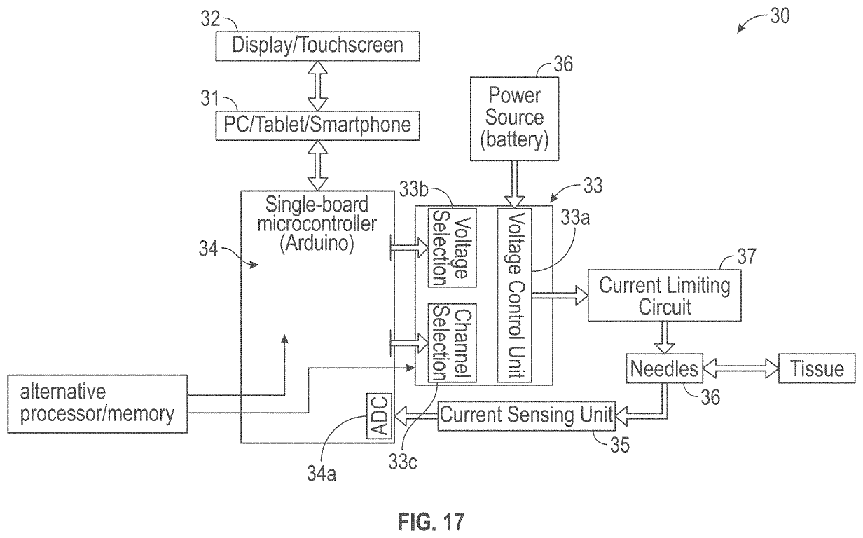

FIG. 17 is a block diagram of an exemplary system for ELF in accordance with the present invention. According to this exemplary embodiment, a system 30 may include a computer 31 with a display 32, which can communicate with a controller 34. In turn, the controller 34 may control a circuit 33 that can include a voltage control unit 33a, a voltage selection unit 33b, and a channel selection unit 33c. For example, the voltage selection unit 33b may enable a user to select a voltage to be applied to a current limiting circuit 37 described below, while the channel selection unit 33c may enable the user to select one or more electrode pairs to be activated in the tissue.

The system 30 may further include a power source 36 may supply power, via the voltage control unit 33a, to a current limiting circuit 37. In turn, the current limiting circuit 37 can apply a potential across cathode and anode needles 36. A current sensing unit or circuit 35 can monitor the current across the needles and provide feedback information, via an analog to digital converter 34a, to the controller 34.

Though an embodiment of the present invention is described in the context of wired circuitry, the present invention contemplates that the same can be implemented in software.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable media having computer readable program code embodied thereon.

Any combination of one or more computer readable storage media may be utilized. A computer readable storage medium is an electronic, magnetic, optical, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer readable storage medium is any tangible medium that can store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable storage medium that can direct a computer, other programmable data processing apparatus, or other devices to function in a particular manner, such that the instructions stored in the computer readable storage medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

It should be understood, of course, that the foregoing relates to exemplary embodiments of the invention and that modifications may be made without departing from the spirit and scope of the invention as set forth in the following claims.

### REFERENCES CITED

(1) Vitale, S.; Sperduto, R. D.; Ferris, F. L. Increased Prevalence of Myopia in the United States Between 1971-1972 and 1999-2004. Arch. Ophthalmol. 2009, 127 (12), 1632-1639. https://doi.org/10.100¹/ₐrchophthalmo1.2009.303.
(2) Holden, B. A.; Fricke, T. R.; Wilson, D. A.; Jong, M.; Naidoo, K. S.; Sankaridurg, P.; Wong, T. Y.; Naduvilath, T. J.; Resnikoff, S. Global Prevalence of Myopia and High Myopia and Temporal Trends from 2000 through 2050. Ophthalmology 2016, 123 (5), 1036-1042. https://doi.org/10.1016/j.ophtha.2016.01.006.
(3) Randleman, J. B. Post-Laser in-Situ Keratomileusis Ectasia: Current Understanding and Future Directions. Curr. Opin. Ophthalmol. 2006, 17 (4), 406-412. https://doi.org/10.1097/01.icu.0000233963.26628.f0.
(4) Hunter, B. M.; Kallick, J.; Kissel, J.; Herzig, M.; Manuel, C.; Protsenko, D.; Wong, B. J. F.; Hill, M. G. Controlled-Potential Electromechanical Reshaping of Cartilage. Angew. Chem. Int. Ed. 2016, 55 (18), 5497-5500. https://doi.org/10.1002/anie.201600856.
(5) Melanie Corbett, Nicholas Maycock, Emanuel Rosen, David O'Brart. Corneal Topography, Principals and Applications, Second Edition.; Springer, 2019.
(6) Panozzo, G.; Mercanti, A. Optical Coherence Tomography Findings in Myopic Traction Maculopathy. Arch. Ophthalmol. 2004, 122 (10), 1455-1460. https://doi.org/10.1001/archopht.122.10.1455.
(7) Romero-Jiménez, M.; Santodomingo-Rubido, J.; Wolffsohn, J. S. Keratoconus: A Review. Contact Lens Anterior Eye 2010, 33 (4), 157-166. https://doi.org/10.1016/j.clae.2010.04.006.
(8) Villa, C.; Gutiérrez, R.; Jimenez, J. R.; Gonzalez-Méijome, J. M. Night Vision Disturbances after Successful LASIK Surgery. Br. J. Ophthalmol. 2007, 91 (8), 1031. https://doi.org/10.1136/bjo.2006.110874.
(9) Pollhammer, M.; Cursiefen, C. Bacterial Keratitis Early after Corneal Crosslinking with Riboflavin and Ultraviolet-A. J. Cataract Refract. Surg. 2009, 35 (3), 588-589. https://doi.org/10.1016/j.jcrs.2008.09.029.
(10) Rama, P.; Di Matteo, F.; Matuska, S.; Paganoni, G.; Spinelli, A. Acanthamoebakeratitis with Perforation after Corneal Crosslinking and Bandage Contact Lens Use. J. Cataract Refract. Surg. 2009, 35 (4), 788-791. https://doi.org/10.1016/j.jcrs.2008.09.035.
(11) Kymionis, G. D.; Portaliou, D. M.; Bouzoukis, D. I.; Suh, L. H.; Pallikaris, A. I.; Markomanolakis, M.; Yoo, S. H. Herpetic Keratitis with Iritis after Corneal Crosslinking with Riboflavin and Ultraviolet A for Keratoconus. J. Cataract Refract. Surg. 2007, 33 (11), 1982-1984. https://doi.org/10.1016/j.jcrs.2007.06.036.
(12) FDA SUMMARY OF SAFETY AND EFFECTIVENESS DATA: Paflufocon B and Paflufocon D Rigid Gas Permeable Contact Lenses.
(13) Wollensak, G.; Spoerl, E.; Seiler, T. Riboflavin/Ultraviolet-a—Induced Collagen Crosslinking for the Treatment of Keratoconus. Am. J. Ophthalmol. 2003, 135 (5), 620-627. https://doi.org/10.1016/S0002-9394(02)02220-1.
(14) Ho, K.-H. K.; Valdes, S. H. D.; Protsenko, D. E.; Aguilar, G.; Wong, B. J. F. Electromechanical Reshaping of Septal Cartilage. The Laryngoscope 2003, 113 (11), 1916-1921. https://doi.org/10.1097/00005537-200311000-00011.
(15) Yau, A. Y. Y.; Manuel, C.; Hussain, S. F.; Protsenko, D. E.; Wong, B. J. F. In Vivo Needle-Based Electromechanical Reshaping of Pinnae: New Zealand White Rabbit Model. JAMA Facial Plast. Surg. 2014, 16 (4), 245-252. https://doi.org/10.1001/jamafacia1.2014.85.
(16) Oswal, V.; Remacle, M.; Jovanvic, S.; Zeitels, S. M.; Krespi, J. P.; Hopper, C. Principles and Practice of Lasers in Otorhinolaryngology and Head and Neck Surgery; Kugler Publications, 2014.
(17) Wormald, J. C. R.; Fishman, J. M.; Juniat, S.; Tolley, N.; Birchall, M. A. Regenerative Medicine in Otorhinolaryngology. J. Laryngol. Otol. 2015, 129 (8), 732-739. https://doi.org/10.1017/50022215115001577.
(18) Loeser, R. F. Molecular Mechanisms of Cartilage Destruction: Mechanics, Inflammatory Mediators, and Aging Collide. Arthritis Rheum. 2006, 54 (5), 1357-1360. https://doi.org/10.1002/art.21813.
(19) Hanson, J. T. Netter's Clinical Anatomy—4th Edition https://www.elsevier.com/books/netters-clin ical-anatomy/hansen/978-0-323-53188-7 (accessed Feb. 19, 2020).
(20) Maurice, D. M. The Structure and Transparency of the Cornea. J. Physiol. 1957, 136 (2), 263-286.1.
(21) Meek, K. M.; Boote, C. The Organization of Collagen in the Corneal Stroma. Exp. Eye Res. 2004, 78 (3), 503-512. https://doi.org/10.1016/j.exer.2003.07.003.
(22) Qu, Y.; Ma, T.; He, Y.; Zhu, J.; Dai, C.; Yu, M.; Huang, S.; Lu, F.; Shung, K. K.; Zhou, Q.; et al. Acoustic Radiation Force Optical Coherence Elastography of Corneal Tissue. IEEE J. Sel. Top. Quantum Electron. Publ. IEEE Lasers Electro-Opt. Soc. 2016, 22 (3). https://doi.org/10.1109/JSTQE.2016.2524618.
(23) Fuentes, M. del C.; Perez, N.; Ayerdi, I. Metallization and Electrical Characterization of Platinum Thin Film Microelectrodes on Biocompatible Polydimethylsiloxane Substrates for Neural Implants. Thin Solid Films 2017, 636, 438-445. https://doi.org/10.1016/j.tsf.2017.06.017.
(24) Dini, J. W.; Johnson, H. R. Plating on Stainless Steel Alloys; United States, 1981; p 13.
(25) Lyons, E. H.; Dempsey, M and R. Electrodeposition of the Platinum Metals. U.S. Pat. No. 3,351,541, 1967.
(26) Zuppardo M. Optical Principles and Light Theories of the Actual EDOF IOL for Presbyopia Correction I.
(27) Salerno, L. C.; Tiveron, M. C., Jr; Alio, J. L. Multifocal Intraocular Lenses: Types, Outcomes, Complications and How to Solve Them. Taiwan J. Ophthalmol. 2017, 7 (4), 179-184. https://doi.org/10.4103/tjo.tjo_19_17.
(28) Naegeli, R.; Redepenning, J.; Anson, F. C. Influence of Supporting Electrolyte Concentration and Composition on Formal Potentials and Entropies of Redox Couples Incorporated in Nafion Coatings on Electrodes. J. Phys. Chem. 1986, 90 (23), 6227-6232. https://doi.org/10.1021/j100281a032.
(29) Kneeland, J. B.; Reddy, R. Frontiers in Musculoskeletal MRI: Articular Cartilage. J. Magn. Reson. Imaging 2007, 25 (2), 339-344. https://doi.org/10.1002/jmri.20811.
(30) Freedman, J. D.; Lusic, H.; Wiewiorski, M.; Farley, M.; Snyder, B. D.; Grinstaff, M. W. A Cationic Gadolinium Contrast Agent for Magnetic Resonance Imaging of Cartilage. Chem. Commun. Camb. Engl. 2015, 51 (56), 11166-11169. https://doi.org/10.1039/c5cc03354c.
(31) Du, Y.; Taga, A.; Suzuki, S.; Liu, W.; Honda, S. Colominic Acid: A Novel Chiral Selector for Capillary Electrophoresis of Basic Drugs. J. Chromatogr. A 2002, 962 (1), 221-231. https://doi.org/10.1016/S0021-9673(02)00593-9.
(32) Crank, J. The Mathematics of Diffusion, Clarendon Press: Oxford.; 1956.
(33) Choi, P.; Jalani, N. H.; Datta, R. Thermodynamics and Proton Transport in Nafion—II. Proton Diffusion Mechanisms and Conductivity. J. Electrochem. Soc. 9.
(34) Park, H. J.; Lim, C. S.; Kim, E. S.; Han, J. H.; Lee, T. H.; Chun, H. J.; Cho, B. R. Measurement of PH Values in Human Tissues by Two-Photon Microscopy. Angew. Chem. Int. Ed. 2012, 51 (11), 2673-2676. https://doi.org/10.1002/anie.201109052.
(35) Wood, P. M. The Potential Diagram for Oxygen at PH 7. Biochem. J. 1988, 253 (1), 287-289.
(36) Kissinger, P. T.; Heineman, W. R. Laboratory Techniques in Electroanalytical Chemistry; 1984.

We claim:

1. A method of altering corneal tissue, comprising:
creating an electrochemical reaction in the tissue;
wherein the electrochemical reaction occurs in the presence of an electrolytic solution in or on the tissue.

2. The method of claim 1, further comprising providing feedback control of an electronic circuit for creating the electrochemical reaction.

3. The method of claim 2, wherein the electronic circuit is one of a potentiostat, a galvanostat, and DC circuit connected to a power supply.

4. The method of claim 1, further comprising altering a pH in at least one of the tissue, tissue internal stress field, and tissue matrix composition.

5. The method of claim 1, further comprising placing at least one electrode in contact with the tissue.

6. The method of claim 5, wherein placing the at least one electrode is in an absence of a voltage gradient across the tissue.

7. The method of claim 1, further comprising disrupting an ionic-bonding network in the tissue.

8. The method of claim 1, further comprising equilibrating the tissue to physiological pH.

9. The method of claim 1, further comprising applying the electrolytic solution or gel on or in the tissue.

10. The method of claim 1, further comprising setting a concentration of electrochemically generated chemical agents that affect the tissue.

11. The method of claim 1, wherein creating the electrochemical reaction includes employing potential-driven electrochemical modification of tissue (PDEMT).

12. The method of claim 1, further comprising:
identifying and isolating at least one discrete electrochemical reaction that causes at least one of shape change in the tissue, change in tissue mechanics, change in tissue viability, change in tissue matrix structure, and change in tissue composition.

13. The method of claim 1, wherein creating the electrochemical reaction is based on at least one of specific electrical dosimetry, electrode placement, electrode geometry configuration, numbers of electrodes, and type of electrode composition.

14. The method of claim 1, further comprising changing at least one of a physical property and a biological behavior of the tissue.

15. The method of claim 14, wherein:
changing the physical property of the tissue includes mechanical behavior—static or dynamic—electrical behavior, optical property, and/or thermal properties; and
changing the biological behavior of the tissue includes tissue viability, matrix structure, and composition.

16. The method of claim 1, further comprising:
providing an working electrode, a reference electrode, and an auxiliary electrode; and
placing the electrodes in a geometric arrangement in the tissue effective for treating or shaping the tissue.

17. The method of claim 16, further comprising mechanically deforming the cornea with a firm hemispherical shell of specific radius of curvature or pre-defined geometry.

18. The method of claim 17, wherein the electrodes are printed, lithographed, etched, or bonded to the shell.

19. The method of claim 18, wherein the electrodes are metallic, polymeric, semi-conductor solid, liquid or gel.

20. The method of claim 17, further comprising mechanically deforming the cornea by a complex geometric shaped shell creating a unique curvature to the cornea to create a user defined corneal shape to produce uni-focal, multi-focal, visual foci or correct astigmatism or aspherical changes.

21. The method of claim 17, wherein the shell is made of non-conductive material that is transparent or opaque.

22. The method of claim 3, further comprising using two or more electrodes as anode or cathode, or working electrode, or counter electrode, or reference electrode.

23. The method of claim 1, further comprising using an additional shell, mandrel, jig made of durable thin material, and a contact lens to apply to the cornea for a period of time after electrochemical treatment.

24. The method of claim 23, wherein the contact lens has sensing circuitry to monitor biophysical properties of the cornea.

25. The method of claim 13, further comprising inserting sensing devices into the shell to monitor stress, pH, optical property changes of the cornea, temperature, or hydration status, which devices are in indirect contact or near contact with the cornea surface.

26. The method of claim 23, further comprising applying a conductive fluid, gel, or paste between the cornea and the shell.

27. The method of claim 1, further comprising shaping of the cornea without the mechanical deformation of the cornea during treatment, and using only the direct application of insertion of electrodes alone.

28. The method of claim 1, further comprising electrochemical activation of an exogenous substrate applied to a surface of the cornea.

29. The method of claim 1, further comprising generating reactive chemical species which lead to cross-linking of corneal matrix proteins.

30. The method of claim 1, further comprising equilibrating the tissue to physiological pH.

31. The method of claim 1, further comprising:
identifying and isolating at least one discrete electrochemical reaction that cause at least one of shape change in the tissue, change in tissue mechanics, change in tissue viability, change in tissue matrix structure, and change in tissue composition.

32. The method of claim 1, further comprising:
providing a working electrode, a reference electrode, and an auxiliary electrode; and
placing the electrodes in a geometric arrangement in contact with, on top of, or within the tissue effective for treating or shaping the tissue.

33. A method of altering corneal tissue, comprising:
using at least an anodic electrode and a cathodic electrode in the tissue to initiate an electrochemical reaction in the tissue;
adding an electrolytic solution to the tissue.

34. The method of claim 33, wherein the method is carried out in the absence of incising the tissue.

35. The method of claim 33, further comprising applying an electrical potential to the tissue.

36. The method of claim 33, further comprising injecting the electrolytic solution into the tissue prior to applying the electrical potential.

37. The method of claim 33, further comprising injecting the electrolytic solution into the tissue while applying the electrical potential.

38. The method of claim 33, wherein the electrolytic solution is saline.

39. The method of claim 33, wherein the electrolytic solution includes an amphiphilic compound.

40. The method of claim 33, further comprising hydrolyzing the tissue.

41. The method of claim 33, further comprising disrupting cell membranes of the tissue.

42. The method of claim 33, further comprising enabling resorption of unwanted tissue into a human.

43. A method of altering corneal tissue, comprising:
mechanically disrupting the tissue; and
electrochemically degrading the tissue.

44. The method of claim 43, wherein mechanically disrupting includes inserting electrodes into the tissue.

45. The method of claim 43, wherein electrochemically degrading includes applying an electrical potential across electrodes in the tissue.

46. A method of altering corneal tissue, comprising:
electrochemically generating sodium hydroxide, hydrogen gas, and either chlorine gas or oxygen gas in the tissue; and
electrochemically forming acid/base species in the tissue.

47. The method of claim 46, wherein electrochemically generating occurs at a negative electrode and a positive electrode in the tissue.

48. The method of claim 46, wherein electrochemically forming includes applying an electrical potential across electrodes in the tissue.

49. Apparatus for altering corneal tissue, comprising:
an electronic system configured to cause an electrochemical reaction in the tissue;
wherein the electrochemical reaction occurs in the presence of native tissue water or an electrolytic solution.

50. The apparatus of claim 49, wherein the electronic system is further configured to select a spatial location of the electrochemical reaction in the tissue.

51. The apparatus of claim 49, wherein the electronic system is further configured to spatially separate an oxidation reaction from a reduction reaction of the electrochemical reaction.

52. The apparatus of claim 49, wherein the electronic system is further configured to produce an electrical potential in the tissue.

53. The apparatus of claim 49, wherein the electronic system includes:
an anode electrode; and
a cathode electrode;
wherein the electrodes are configured to be placed on top at the surface or inserted into the tissue.

54. The apparatus of claim 49, wherein the electronic system includes:
a power source;
a voltage control unit powered by the power source; and
a current limiting circuit in communication with the voltage control unit.

55. The apparatus of claim 49, wherein the electronic system includes:
a current limiting circuit; and
at least one electrode in communication with the current limiting circuit.

56. Apparatus for altering corneal tissue, comprising:
at least two electrodes configured to cause an electrochemical reaction in the tissue;
wherein the electrochemical reaction occurs in the presence of an electrolytic solution; and
a controller in communication with the electrodes and configured to:
create an electrical potential across the electrodes; and
cause an oxidation reaction spatially distinct in the tissue from a reduction reaction in the tissue.

57. The apparatus of claim 56, wherein the controller is further configured to control a voltage control unit in communication with the at least two electrodes.

58. The apparatus of claim 56, wherein the controller is further configured to communicate with a computer.

59. The apparatus of claim 56, wherein the controller is further configured to control a current limiting circuit in communication with the electrodes.

60. The apparatus of claim 56, wherein the controller is further configured to control a current sensing unit in communication with the electrodes.

61. A computer-implemented method for altering corneal tissue, comprising:
initiating, by a processor, an electrochemical reaction in the tissue;
wherein the electrochemical reaction occurs in the presence of an electrolytic solution in the tissue.

62. The method of claim 61, further comprising initiating, by the processor, an electrical potential in the tissue.

63. The method of claim 61, further comprising controlling, by the processor, a non-linear electronic circuit to produce the electrochemical reaction.

64. A non-transitory computer readable medium with computer executable instructions stored thereon, executed by a processor, to perform a method for altering corneal tissue, the method comprising:
inducing an electrochemical reaction in the tissue;
wherein the electrochemical reaction occurs in the presence of an electrolytic solution in the tissue.

65. The method of claim 64, wherein the processor controls a battery to create the electrochemical reaction.

66. The method of claim 64, wherein the processor controls a potentiostat to create the electrochemical reaction.

67. The method of claim 64, wherein the processor controls a galvanostat to create the electrochemical reaction.

68. The method of claim 1, further comprising providing feedback control of an electronic circuit for creating the electrochemical reaction.

69. The method of claim 68, wherein the electronic circuit is one of a potentiostat, a galvanostat, and DC circuit connected to a power supply.

70. The method of claim 1, further comprising disrupting cell membranes of the tissue.

71. The method of claim 1, further comprising enabling resorption of unwanted tissue into a human.

72. An apparatus to reshape the corneal tissue, comprising:
means for creating stress in the tissue to temporarily define and maintain a predetermined shape of the tissue;
means for monitoring the internal stresses, geometry, pH, optical clarity, and temperature of the tissue;
means for causing a direct current of a predetermined polarity to flow in the tissue to mediate the tissue while the created stress is present to permanently change shape of the tissue or material parameters of the tissue without ablation or carbonization; and
means for controlling the direct current flowing in the tissue according to the stresses therein.

73. An apparatus of reshaping tissue to reshape the tissue, comprising:
means for creating stress in the tissue to temporarily define and maintain a predetermined shape of the tissue;
means for causing a direct current of a predetermined polarity to flow in the tissue;
means for applying voltage pulses of the same polarity to form a DC pulse train to mediate the tissue while the created stress is present to permanently change shape of the tissue or material parameters of the tissue without carbonization or ablation; and
means for applying a voltage of predetermined polarity to obtain a predetermined bioeffect;
means for applying a first sequence of voltage pulses of the same polarity and
means for applying a second sequence of voltage pulses of the opposite polarity or same polarity with different magnitude to form a complex DC pulse train.

74. An apparatus of electroforming tissue to reshape the tissue, comprising:
means for creating stress in the tissue to temporarily define and maintain a predetermined shape of the tissue;
means for causing a direct current of a predetermined polarity to flow in the tissue;
means for applying voltage pulses of the same polarity to form a DC pulse train to mediate the tissue while the created stress is present to permanently change shape of the tissue or material parameters of the tissue without carbonization or ablation;
means for applying a voltage of predetermined polarity to obtain a predetermined bioeffect;
means for applying a first sequence of voltage pulses of the same polarity;
means for applying a second sequence of voltage pulses of the opposite polarity to form a complex DC pulse train;
means for applying a first sequence; and
means for applying a second sequence of voltage pulses provide a net charge cancellation when integrated over an application time.

75. A method of reshaping corneal tissue, comprising:
direct application of pressure on the tissue to cause a predetermined shape of the tissue; and
optionally, applying a negative pressure on the tissue to pull the tissue towards a jig.

76. The method of claim 1, further comprising using an electrode geometry that is complex in three dimensions.
